(19) United States
(12) Patent Application Publication
(10) Pub. No.: US 2026/0218110 A1
(43) Pub. Date: Jul. 30, 2026

(54) SYSTEMS AND METHODS OF BIOMASS MONITORING FOR CELL CULTURE BIOREACTORS

(71) Applicant: CORNING INCORPORATED, CORNING, NY (US)

(72) Inventors: Ye Fang, Painted Post, NY (US); Kathleen Anne Krebs, Painted Post, NY (US); Yujian Sun, Painted Post, NY (US)

(21) Appl. No.: 19/144,787

(22) PCT Filed: Dec. 21, 2023
(86) PCT No.: PCT/US2023/085280

§ 371 Date: Jun. 30, 2025

Related U.S. Application Data
(60) Provisional application No. 63/436,310, filed on Dec. 30, 2022.

(51) Int. Cl. C12M 1/34; C12M 1/00; C12M 1/12

(52) U.S. Cl. CPC C12M 41/36; C12M 25/18; C12M 29/10; C12M 41/32; C12M 41/46

(57) ABSTRACT

A method of monitoring biomass during a cell culture of cells in a bioreactor is provided. The method includes culturing the cells in the bioreactor using a cell culture medium perfused through the bioreactor; measuring at least one of a cell nutrient and a cell byproduct in the cell culture medium; determining at least one of a consumption rate of the cell nutrient and an accumulation rate of the cell byproduct; and predicting a cell number within the bioreactor at a specified culture time based on at least one of the consumption rate and the accumulation rate.

### CROSS-REFERENCE TO RELATED APPLICATIONS

This application claims the benefit of priority under 35 U.S.C. § 119 of U.S. Provisional Application Ser. No. 63/436,310 filed on Dec. 30, 2022, the content of which is relied upon and incorporated herein by reference in its entirety.

### FIELD OF THE DISCLOSURE

This disclosure general relates to systems and methods of monitoring cell cultures in bioreactor systems. In particular, the present disclosure relates to methods, protocols, systems, and models for biomass monitoring of a cell culture within a bioreactor system.

### BACKGROUND

In the bioprocessing industry, large-scale cultivation of cells is performed for purposes of the production of hormones, enzymes, antibodies, vaccines, therapeutic proteins, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and other multilayered vessels (e.g., the HYPERStack® from Corning Inc.). These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks; hollow fiber bioreactors, in which cells may form large three-dimensional aggregates as they proliferate in the interspatial fiber space; and packed-bed bioreactors. In packed-bed or fixed-bed bioreactors, a packed or fixed cell substrate is used to provide a surface for the attachment of adherent cells. Medium is perfused along the surface or through the semi-porous substrate to provide nutrients and oxygen needed for the cell growth. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer.

One of the significant issues with traditional fixed bed bioreactors is the non-uniformity of cell distribution inside the bed. For example, the packed bed can function as a depth filter with cells predominantly trapped at the inlet regions or other regions of relatively low flow and/or high substrate density, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Another significant drawback of traditional packed bed systems disclosed in a prior art is the inability to efficiently harvest intact viable cells at the end of culture process. Harvesting of cells is important if the end product is cells, or if the bioreactor is being used as part of a “seed train,” where a cell population is grown in one vessel and then transferred to another vessel for further population growth. U.S. Pat. No. 9,273,278 discloses a bioreactor design to improve the efficiency of cell recovery from the packed bed during cells harvesting step. It is based on loosening the packed bed matrix and agitation or stirring of packed bed particles to allow porous matrices to collide and thus detach the cells. However, this approach is laborious and may cause significant cells damage, thus reducing overall cell viability.

In addition, because of the random arrangement of fibers in the traditional packed or fixed bed substrates, it can be difficult for bioreactor users to predict cell culture performance, since the substrate arrangement and/or packing varies between cultures. Monitoring of the cell culture health or progress is also difficult. For example, the presence of the fixed bed itself limits options for effectively monitoring the health of the culture and the biomass production. Furthermore, the packed substrate of traditional fixed bed bioreactors makes efficiently harvesting cells very difficult or impossible, as it is believed that cells are entrapped by the packed bed, which further hampers an understanding of the cell culture performance.

Regardless of the platform used, the earlier stages of process development require users to have information for better understanding of cell behavior, virus production, and culture progress. The upstream bioreactor process development requires the identification of critical parameters and quality features as well as the parameter definition and their connection to the final product. Understanding what these parameters are and how they might scale with higher-density or larger systems is important for process development and efficiency.

Upstream bioprocess production also goes through good manufacturing process (GMP) regulations as well as requirements referred to process analytical technology (PAT). PAT is regarded as a tool for the design, analyses and control of production processes. The final product quality can be ensured through the measurement of process parameters and product characteristics. This can include extensive online culture process monitoring, which provides a useful tool for process characterization and the detection of process changes. Relevant parameters for packed bed bioreactor process characterization and control are pH, temperature, dissolved oxygen or oxygen delivery (DO2), and carbon dioxide (CO₂). However, one of the identified drawbacks of packed bed bioreactors is the difficulty in taking substrate samples to directly assess the state of the cells and the overall cell culture progress. Taking substrate samples risk contaminating the entire culture or, in the case of non-uniform platforms, providing misleading or inaccurate data.

Fixed bed bioreactors have been increasingly used for scale-up in adherent cell culture. Biomass monitoring is an important tool to design, analyze, and control manufacturing processes of pharmaceuticals when cell culture is involved. For suspension cell culture, biomass monitoring can be achieved using optical or electric approaches. However, for adherent cell culture using fixed bed bioreactors there are not validated approaches or sensors available for satisfactory online biomass monitoring. Historically, substrate (e.g., glucose) consumption rates have been proposed to be useful for understanding the kinetics of cell growth. However, since glucose consumption rate (GCR) is influenced by many factors besides the viable cell number, there are no established protocols, methods and models to obtain and use effective substrate consumption rates and metabolite accumulation rates for biomass calculation and predictions.

While manufacturing of viral vectors for early-phase clinical trials is possible with existing platforms, there is a need for a platform that can produce high-quality product in greater numbers in order to reach late-stage commercial manufacturing scale. In addition, there is a need for systems and methods that enable collection of specific measurable parameters from the cell culture during a bioreactor run to have better real time control of aspects of the culture process, and to detect and diagnose abnormal culture conditions.

### SUMMARY

According to embodiments of this disclosure, a method of monitoring biomass during a cell culture of cells in a bioreactor is provided. The method includes culturing the cells in the bioreactor using a cell culture medium perfused through the bioreactor; measuring at least one of a cell nutrient and a cell byproduct in the cell culture medium; determining at least one of a consumption rate of the cell nutrient and an accumulation rate of the cell byproduct; and predicting a cell number within the bioreactor at a specified culture time based on at least one of the consumption rate and the accumulation rate. According to an aspect of embodiments, the bioreactor is a fixed bed bioreactor having a substrate configured for culturing cells attached to a surface of the substrate. The at least one cell nutrient can be glucose or glutamine; and the at least one cell byproduct can be lactate or ammonia. The cell culture medium can be a glucose- or a glutamine-rich cell culture medium, according to an aspect of embodiments.

According to aspects of embodiments, the measuring of the at least one of the cell nutrient and the cell byproduct in the cell culture medium includes taking multiple measurements of the cell nutrient or the cell byproduct, the multiple measurements being separated by a measurement interval that is less than a doubling time of the cells in the cell culture. The measurement interval is greater than or equal to a minimum interval time, the minimum interval time being a time at which the change in the level of the cell nutrient or the cell byproduct is larger than a measurement tolerance of measuring the cell nutrient or cell byproduct. In aspects of embodiments, the minimum interval time is greater than or equal to about 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, or 20 hours. The measuring of the cell nutrient in the cell culture medium can include measuring the cell nutrient multiple times per day of the cell culture. The measuring of the cell byproduct in the cell culture medium can include measuring the cell byproduct multiple times per day of the cell culture.

According to aspects of embodiments, predicting the cell number includes using a mathematical model to calculate biomass at the specified culture time. The measuring can include using an inline sensor in a perfusion line of the cell culture medium. The measuring can also include using an offline measurement of samples of the cell culture medium.

According to aspects of embodiments, the method further includes, after the determining of the consumption rate and the accumulation rate, comparing at least one of the consumption rate of a first cell nutrient and the accumulation rate of a first cell byproduct to at least one of the consumption rate of a second cell nutrient and the accumulation rate of a second cell byproduct. The comparing can include comparing the consumption rate of glucose to the consumption rate of glutamine, comparing the consumption rate of glucose to the accumulation rate of ammonia, comparing the accumulation rate of lactate to the consumption rate of glutamine, and/or comparing the accumulation rate of lactate to the accumulation rate of ammonia. The method can further include determining an abnormality in the cell culture based on the comparing. The method can further include seeding cells in the bioreactor at a seeding density. The method can further include supplying fresh cell culture medium to the bioreactor, and the measuring can include a first measurement, the first measurement occurring at least one hour after supplying the fresh cell culture medium. According to aspects of embodiments, the predicting of the cell number, Nₜ, includes using the following equation:

$${N}_{t} = {N}_{seed} {e}^{kt}$$

where N_{seed} is the number of cells seeded into the bioreactor, k is a cell growth rate, and tis the time for which the cell number is being predicted.

### BRIEF DESCRIPTION OF THE DRAWINGS

FIG. 1 is a schematic representation of a cell culture system, according to one or more embodiments.

FIG. 2 shows an operation for controlling a perfusion flow rate of a cell culture system, according to one or more embodiments.

FIG. 3 is a graph of bioreactor perfusion flow rate and oxygen concentration over time during an example bioreactor run using a bioreactor system according to FIG. 1, according to one or more embodiments.

FIG. 4A is a graph of the dissolved oxygen concentration over time during the bioreactor run of FIG. 3.

FIG. 4B is a graph of the pH over time during the bioreactor run of FIG. 3.

FIG. 4C is a graph of the media conditioning temperature over time during the bioreactor run of FIG. 3.

FIG. 5 is a graph of the oxygen consumption of the packed bed cell culture over time during the bioreactor run of FIG. 3, including the slope α of the curve.

FIG. 6 is a graph of the slope α versus cell seeding density of the bioreactor, according to one or more embodiments.

FIG. 7 is a graph of the slope α versus cell culture substrate surface area, according to one or more embodiments.

FIG. 8 is a graph of the cell harvest yield versus the slope α, according to one or more embodiments.

FIG. 9 is a graph the concentration profile of glucose through four days of an experimental culture, according to embodiments.

FIG. 10 is a graph of the concentration profile of lactate through four days of an experimental culture, according to embodiments.

FIG. 11 is a graph of the total amount of glucose consumed, V(C₀−Cₜ), as a function of time each day, according to example embodiments.

FIG. 12 is a graph of the total glucose consumption rate, rN₀, at each day as an exponential function over culture duration, according to example embodiments.

FIG. 13 is a diagram of an experimental setup, according to embodiments.

FIG. 14A is a graph of the total glucose consumption rate, rN₀, at each day as an exponential function over culture duration, according to embodiments.

FIG. 14B is a graph of the total glutamine consumption rate, rN₀, at each day as an exponential function over culture duration, according to embodiments.

FIG. 14C is a graph of the total lactate accumulation rate, rN₀, at each day as an exponential function over culture duration, according to embodiments.

FIG. 14D is a graph of the total ammonia accumulation rate, rN₀, at each day as an exponential function over culture duration, according to embodiments.

FIG. 15 is a graph showing a plot of glucose consumption over time as it fits to an exponential model, according to embodiments.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure include systems and methods for monitoring and controlling the cell culture. This disclosure describes systems and methods to collect specific signal signatures during bioreactor run to have better real time control of critical aspects and to detect and diagnose abnormal culture conditions. Identified signature parameters of the cell culture described in this disclosure can be used as a tool for process analytical technology implementation and for online monitoring of upstream process. As a result, the optimized cell culture production processes can be established by development of routine and reproducibility of the signature operating parameters.

According to embodiments of this disclosure, bioreactor systems and methods are provided for monitoring the state of a cell culture in the bioreactor system during a cell culture run. In particular, embodiments describe a bioreactor system having an outlet sensor at the outlet of a cell culture bioreactor or vessel, as well as systems capable of real-time signal collection and real-time process of signals from this and/or other sensors, and methods of cell culture using such systems. For example, methods include using such sensor signals as trigger points for important cell culture process steps, or for predicting the expected or assessing the current health of a cell culture for a certain bioreactor size or seeding density over time. The advantages of these systems and methods include the ability to actively monitor the bioreactor state in real time without the need to perform physical sampling of the packed bed substrate for off-line analysis. Continuous monitoring of bioreactor state will also allow end users to actively adjust the bioprocess steps that are dependent on the progression of culture processes inside the packed bed bioreactor. The ability to characterize and log the progression of bioprocess run will further allow end users to monitor and record batch to batch consistency of the process. This type of tracking of progression and consistency among cell culture runs can be incredibly advantageous.

In conventional large-scale cell culture bioreactors, different types of packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped or entangled in fibers of the matrix. Thus, because of linear flow of the cell inoculum through the packed bed, cells are subject to heterogeneous distribution inside the packed-bed, leading to variations in cell density through the depth or width of the packed bed. For example, cell density may be higher at the inlet region of a bioreactor and significantly lower nearer to the outlet of the bioreactor. In another example, non-uniformities in the packed bed create a channeling effect in which cell culture media preferentially flows in certain areas of the bed while be restricted from reaching other areas of the bed, again leading to non-uniform cell distribution and nonuniform or inconsistent medium or nutrient distribution. This non-uniform distribution of the cells inside of the packed bed significantly hinders scalability and predictability of such bioreactors in bioprocess manufacturing, and can even lead to reduced efficiency in terms of growth of cells or viral vector production per unit surface area or volume of the packed bed.

Another problem encountered in packed-bed bioreactors disclosed in prior art is the channeling effect, described above. Due to random nature of packed nonwoven fibers, the local fiber density at any given cross section of the packed bed is not uniform. Medium flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide bioreactor systems, cell growth substrates, matrices of such substrates, and methods using such bioreactor systems and substrates that enable efficient and high-yield cell culturing for anchorage-dependent cells and production of cell products (e.g., proteins, antibodies, viral particles). Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, productions is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE™ (produced by Thermo Fisher Scientific), or Accutase® (produced by Innovative Cell Technologies).

According to one or more embodiments, a cell culture bioreactor can include a cell culture substrate within the bioreactor vessel. The substrate can be deployed in a packed bed bioreactor configuration, or in other configurations within a three-dimensional culture chamber of the bioreactor vessel. Due to contamination concerns, the vessel can be a single-use vessel that can be disposed of after use.

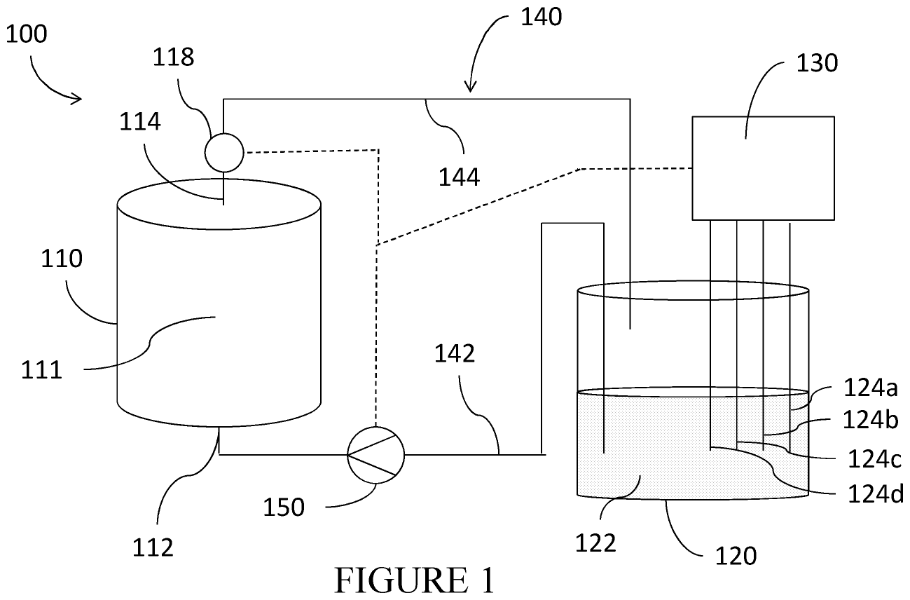

As shown in FIG. 1, embodiments of this disclosure include a bioreactor system 100 for culturing cells in a cell culture vessel 100. The cell culture vessel includes an inlet 112 and an outlet 114 that are fluidly connected to an interior reservoir 111 of the cell culture vessel 110. The interior reservoir 111 contains a space for containing and culturing cells, and may also include a cell culture substrate (not shown) on which adherent-based cells can be cultured. In some embodiments, the inlet 112 is located at one end of the cell culture vessel 110 for the input of media, cells, and/or nutrients into the cell culture vessel 110, and the outlet 114 is located at the opposite end for removing media, cells, or cell products from the cell culture vessel 110. The substrate within the interior reservoir can take many forms, some of which are discussed herein by way of example. Some embodiments may use one or both of the inlet 112 and outlet 114 for flowing media, cells, or other contents both into and out of the cell culture vessel 110. For example, inlet 112 may be used for flowing media or cells into the cell culture vessel 110. During cell seeding, perfusion, and/or culturing phases, but may also be used for removing one or more of media, cells, or cell products through the inlet 112 in a harvesting phase. Thus, the terms “inlet” and “outlet” are not intended to restrict the function of those openings, but should generally be understood to mean the ports used for inletting and outletting, respectively, of fluid during the regular course of cell growth. An outlet sensor 118 is provided at the outlet 114 of the cell culture vessel 110. As used herein, “at the outlet” can mean a sensor providing in-line of a fluid flow path that receives media from the outlet 114 and returns the media to another part of the system (e.g., a media conditioning vessel), or it can mean a sensor provided within the cell culture vessel 110 but preferably after the cell culture substrate, packed bed, or other cell culture zone within the cell culture vessel 110. In this way, the outlet sensor 114 can detect a property of media after it has passed through the packed bed, cell culture substrate, or other cell culture zone.

The system further includes a media conditioning vessel (MCV) 120 that can hold and condition cell culture media 122. A fluid flow path 142, 144 delivers conditioned media 112 from the MCV 120 to the cell culture vessel 110, and returns used media from the cell culture vessel 110 to the MCV 120. The MCV 120 can be coupled with a plurality of sensors and/or conditioning components 124a, 124b, 124c, 124d used to sense properties of the cell culture media and to adjust or condition that media, as needed during the cell culture. These include but are not limited to dissolved gas (e.g., O₂, air, CO₂, N₂) sensors and supplies, pH sensors, oxygenator/gas sparging unit, temperature probes and temperature control devices, and nutrient addition and base addition ports. A gas mixture supplied to sparging unit can be controlled by a gas flow controller for N₂, O₂, and CO₂ gasses. The media conditioning vessel 120 can also contain an impeller for media mixing.

The system can also include a media conditioning control unit 130, operatively connected to the plurality of sensors and/or conditioning components 124a, 124b, 124c, 124d to process signals detected from those sensors and/or to control the conditioning components to condition the media 122 within the MCV 120. The media conditioning control unit 130 can also be operatively connected to a pump 150 to control the pump 150 and thus control the rate of fluid flow through the fluid flow path 142, 144 and the perfusion through the cell culture vessel 110. Alternatively, the pump 150 and outlet sensor 118 can be connected directly or connected via a perfusion control unit separate from the media conditioning control unit 130. In some embodiments herein, a peristaltic pump is used, but other pump types are possible. As shown in FIG. 1, the media conditioning vessel 120 is provided as a vessel that is separate from the bioreactor vessel 110. This can have advantages in terms of being able to condition the media separate from where the cells are cultured, and then supplying the conditioned media to the cell culture space. However, in some embodiments, media conditioning can be performed within the bioreactor vessel 110.

In some embodiments, the media conditioning control unit 130 can be used to maintain a steady or desired level of various parameters of the cell culture media 122 within the MCV 120, thus maintaining the bulk media 122 at specific temperature, oxygen saturation level, pH, and CO₂ concentration. For example, for a given cell line or stage of the cell culture process, it may be desirable for the cell culture media 122 to have a certain temperature, pH, dissolved gas content, or nutrient level for optimal cell health and/or growth. The media 122 from the media conditioning vessel 120 is delivered to the cell culture vessel 110 via the inlet 112, which may also include an injection port for cell inoculum to seed and begin culturing of cells. The cell culture vessel 110 may also include the outlet 114 through which the cell culture media 122 exits the vessel 110. In addition, cells or cell products may be output through the outlet 114. To analyze the contents of the outflow from the cell culture vessel 110, the outlet sensor 118 is provided. As described above, the media conditioning control unit 130 may receive a signal from the outlet sensor 118 (e.g., an O₂ sensor) and, based on the signal, adjust the fluid flow through the cell culture vessel 110 by sending a signal to a pump 150 (e.g., peristaltic pump) upstream of the inlet 112 of the cell culture vessel 110. Thus, based on one or a combination of factors measured by the outlet sensor 118, the pump 150 can control the flow into the cell culture vessel 110 to obtain the desired cell culturing conditions. Because the cell culture media 122 within the MCV 120 can be maintained at a desired state, the changing of the flow rate can effectively address any need of the cells within the cell culture vessel 110. For example, because cell culture media 122 leaving the MCV 120 is conditioning for optimal performance, the media entering via inlet 112 should meet the optimal requirements for the media. If the outlet sensor 118 detects a less than desirable level in the cell culture media existing the cell culture vessel 110 at the outlet 114, that can mean, for example, that the cells in the culture have consumed some amount of dissolved gas (e.g., oxygen) or cell nutrients in the media, and at least some cells (i.e., those near the outlet where the media is most depleted) are not being cultured optimally. Therefore, if, for example, the level of dissolved oxygen in the cell culture media at the outlet sensor 118 is lower than optimal (e.g., for a given cell type, stage of culture, etc.), the perfusion flow rate can be increased to supply a higher rate of the conditioned media, which should then result in all cells (even those near the outlet) being cultured under optimal conditions.

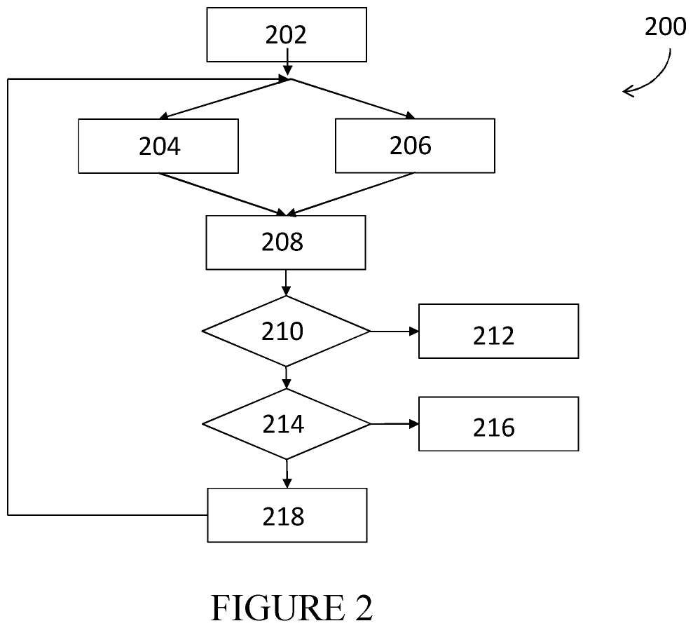

The media perfusion rate is controlled by the media conditioning control unit 130 that collects and compares sensors signals from media conditioning vessel 120 and sensors 124a-124d in the MCV 120, as well as the outlet sensor 118. Because of the pack flow nature of media perfusion through a packed bed substrate in the cell culture vessel 110, nutrients, pH and oxygen gradients are developed along the packed bed. The perfusion flow rate of the bioreactor can be automatically controlled by the media conditioning control unit 130 operably connected to the pump 150. This control scheme is represented in the flow diagram of FIG. 2. In the sensing and control process 200 shown in FIG. 2, at step 202, optimal conditions are predetermined through a round of bioreactor optimization runs. These optimal conditions include minimum pH, minimum oxygen level, and nutrient (e.g., glucose) at the outlet sensor 118, and the pH, oxygen level, and nutrient (e.g., glucose) in the MCV 120. These parameters are provided by way of example, and a person of ordinary skill in the art could understand that other parameters may be relevant for a given application (e.g., temperature). The pH and oxygen levels in the MCV 120 are controlled independently based on inputs from the respective sensors located in the MCV 120. The nutrient (e.g., glucose) level is maintained in the MCV 120 based in part on a signal from the outlet sensor 118, such that the nutrient level in the MCV 120 remains greater than a nutrient level detected by the outlet sensor 118. During the cell culture run, step 204 and 206 are conducted in parallel. In step 204, the outlet sensor 118 is used to measure conditions at the cell culture vessel 110 outlet 114 (e.g., pH, O₂, and glucose). In step 206, sensors 124a-124d are used to measure conditions in the MCV 120 (e.g., pH, O₂, and glucose). In step 208, the perfusion pump 150 is controlled by the control unit based on input from both steps 202 and 204. In step 210, it is determined if the pH at the outlet sensor 118 is greater than the minimum pH determined in step 202; if the oxygen at the outlet sensor 118 is greater than the minimum oxygen level determined in step 202; and if the nutrient level in the MCV 120 is greater than the nutrient level at the outlet sensor 118, and whether the nutrient level at the outlet sensor is greater than the minimum level determined in step 202. If all of these conditions are met, the perfusion by the pump is continued at the present flow rate (step 212). If those conditions are not met, step 214 asks whether the current perfusion rate is less than or equal to the max flow rate. If it is not, then the system reevaluates minimum pH, 02, and glucose at the outlet 114, or increases the nutrient level in the MCV 120. However, if the current perfusion rate is less than the max flow rate, step 218 dictates that the perfusion flow rate be increased. The sensing and control scheme 200 returns to the top of the chart in FIG. 2 for steps 204 and 206.

According to embodiments of this disclosure, it is therefore possible to directly measure nutrient and/or oxygen consumption of cells within the cell culture vessel and to respond in a way that maintains desired conditions for the cells. For example, during the cell culture run, the media conditioning control unit 130 is preprogrammed to maintain a specific level of oxygen saturation in the bulk media volume relative to the atmospheric saturation, where that level in the MCV is measured by the sensors 124a-124d. Placement of second sensor (outlet sensor 118) at the bioreactor outlet 114 measures oxygen saturation level in the media just as it leaves the cell culture vessel. Using these sensors and controls, a constant oxygen depletion level can be maintained within the range of physiological conditions by automatic adjustment of perfusion flow rate.

According to some embodiments of this disclosure, systems and methods are provided for improved process monitoring that can accelerate the process development for cell culture protocols and improve efficiency and reproducibility of the cell culture process. The ability to characterize and log the progression of these bioprocess runs will allow the end user to monitor and record batch to batch consistency of the process. Relevant parameters for the process characterization are cell growth, cell quality, medium conditions (temperature, pH, pO2, and pCO2) as well as metabolite concentrations (glucose, lactate, glutamine and ammonium). Temperature, pH, pO2, and pCO2 of bulk media are routinely controlled online in cell culture, but online monitoring of these and other process parameters in dynamic systems is not done today. Thus, embodiments of this disclosure provide systems and methods to obtain, e.g., the oxygen consumption parameter in a packed bed perfusion bioreactor and demonstrate that such parameter is characteristic for a given bioprocess, and therefore can be used as a signature parameter for a given bioprocess.

As discussed above, FIG. 1 presents a schematic of bioreactor system (e.g., a fixed bed perfusion bioreactor). Cell culture media that enters the cell culture vessel 110 through the inlet 112 can have 100% atmospheric oxygen saturation. Alternatively, according to Henry's law, concentration of a gas in liquid phase is equal to Henry's law constant (k) multiplied by the partial pressure of that gas in the gas phase, therefore oxygen saturation can be presented as concentration of oxygen in cell culture media and equal to 204 μM at 100% saturation at normal atmospheric pressure. During media passage through the cell culture vessel, dissolved oxygen is being used by the immobilized cells and its concentration in the cell culture media drops. Different cell types have different oxygen consumption rates. But a bioreactor system with a sensing and control system of this disclosure allows a user to run the process with specified oxygen concentration at the bioreactor outlet, measured by the outlet sensors 118 and the media conditioning and perfusion control system operates according to logic presented in the flow diagram of FIG. 2.

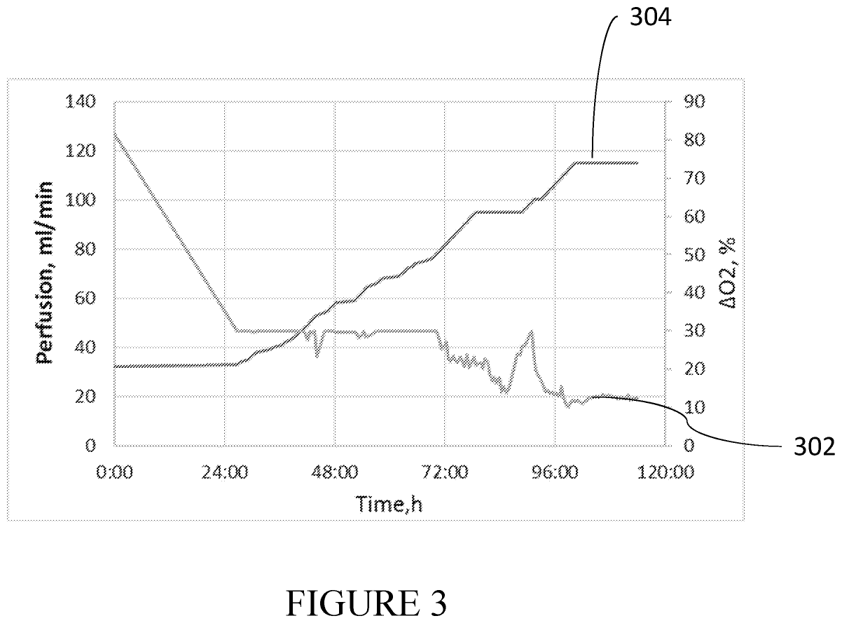

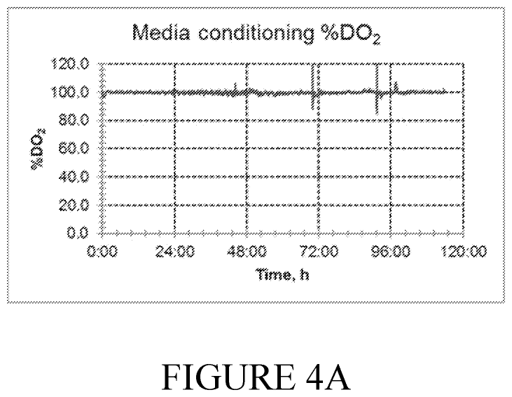

To illustrate this sensing and control of the bioreactor system, some examples will be presented. Specifically, FIG. 3 shows a typical graph of percent dissolved oxygen (302) over time during a bioreactor run as measured by an outlet sensor 118, and the corresponding perfusion rate (304) (ml/min) of the media in the system. Flow rate was controlled automatically by a peristaltic perfusion flow control unit. In this example, the bioreactor is seeded with the cells at time 0:00 hours and the user set a minimal oxygen saturation level of media at the outlet sensor 118 to 30%. The initial media perfusion flow rate was set to 33 ml/min. Inoculation cells are then provided into the bioreactor system and begin to attach to the packed bed substrate and proliferate. Accordingly, oxygen consumption increases and saturation level of the media at outlet decreases to ~30% a.s. at 26 hours post-seeding. As a result, the control system automatically increases the perfusion flow rate to maintain that minimum 30% oxygen saturation level at the bioreactor outlet 114. At 72 hours post-inoculation, the user decreased the setting of the minimum outlet oxygen saturation level from 30% to 15% and the cell culture proceeded in automatic mode. It should be noted that medium conditions were independently maintained in a media conditioning vessel by the media conditioning control unit. Examples of media conditioning vessel parameters are presented in FIGS. 4A, 4B, and 4C. Specifically, FIG. 4A shows the percentage of oxygen in the media of the MCV over time. FIG. 4B shows the pH of that media over time, and FIG. 4C shows the temperature of that media over time.

As mentioned above, embodiments include the real time processing of signals and control of a bioreactor system, and the development of a characteristic signal signature of a specific bioreactor run, which can be used as an analytical tool and to compare and validate independent bioreactor runs. Thus, characteristic signal signature can be used to evaluate the health of cells cultured inside the cell culture vessel and make decisions regarding the next process steps to occur during the bioreactor run. For example, as discussed above, FIG. 3 shows recorded oxygen saturation concentration over time at the bioreactor outlet 114 during the cell culture process. The oxygen concentration level at the bioreactor outlet dropped from ~82% at time point 0 hours to about 30% during the first 26 hours of the bioreactor run. While this occurred, the oxygen concentration at the bioreactor inlet 112 was kept constant, as shown by the value detected in the MCV 120 in FIG. 4A. Using the oxygen concentration at inlet and outlet, an oxygen consumption rate for the cells in culture can be determined using Equation 1:

$$Total oxygen consumption in packed bed ( \frac{\% a . s .}{min} ) = Pump flow rate ( \frac{ml}{min} ) * ( 100 - Oxygen saturation level at bioreactor outlet , \% ) . \qquad Equation ( 1 )$$

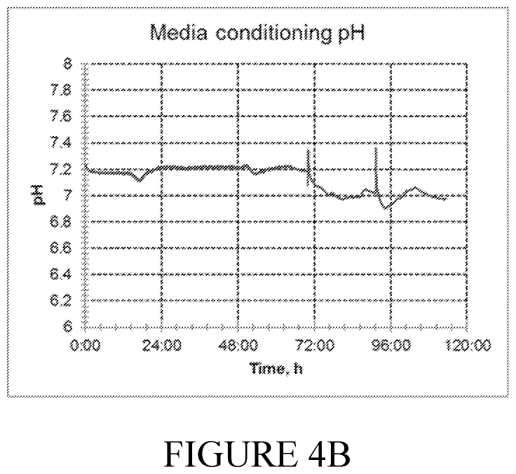

This oxygen consumption rate is shown in FIG. 5 (expressed in % a.s./min) over time (in hours). FIG. 5 also shows a dotted line representing the approximate slope, α, of the line, which can be used as the characteristic signal signature of the bioreactor run. In other words, the value of the data's slope α in FIG. 5 directly reflects the cell culture progression inside the bioreactor system. This value can be used as a process analytical tool to control and describe the upstream bioprocess. The examples below demonstrate that the slope α from graphs similar to FIG. 5 directly relates to the health of the cell culture and from the biomass inside packed bed matrix.

To illustrate the use of the parameter a, multiple cell culture runs were performed using bioreactor systems having differently sized cell culture substrate, bed heights, and cell seeding densities. Table 1 summarizes parameters of the seven cell culture runs used.

TABLE 1
Summary of 7 bioreactor system runs, including height of the packed bed (cm), total
cells seeded (millions of cells), total packed bed surface area (cm²), seeding density
(cells/cm²), total harvested cells (billions of cells), viability of harvested cells (%), harvested

| | | | Total | | | | | | | |
|---|---|---|---|---|---|---|---|---|---|---|
| | Height | Total | packed | | Total | | | | | |
| | of the | cells | bed | | harvested | | Harvested | Max. | | |
| | packed | seeded, | surface | Seed | cells, | | density, | perfusion | Max. O₂ | |
| | bed, | millions | area, | density, | billions | | thousand | flow, | consumption, | Slope |
| # | cm | cells | cm² | cells/cm² | cells | Viability | cells/cm² | ml/min | AU | α |
| 1 | 2.7 | 151 | 6780 | 22,222 | 3.12 | 98% | 460K | 91 | 5400 | 42 |
| 2 | 2.7 | 151 | 6780 | 22,222 | 2.47 | 92% | 364K | 100 | 6000 | 34.62 |
| 3 | 2.7 | 151 | 6780 | 22,222 | 1.68 | 96% | 248K | 70 | 4000 | 19 |
| 4 | 5.4 | 302 | 13560 | 22,227 | 7.20 | 92% | 530K | 115 | 10051 | 70.13 |
| 5 | 5.4 | 302 | 13560 | 22,227 | 6.10 | 88% | 449K | 90 | 7700 | 72.9 |
| 6 | 5.4 | 302 | 13560 | 22,227 | 7.08 | 92% | 522K | 130 | 10000 | 73.6 |
| 7 | 8.1 | 453 | 20340 | 22,222 | 9.20 | 95% | 452K | 200 | 16815 | 92.75 |

density (cells/cm²), maximum perfusion flow rate (ml/min), maximum O₂ consumption (AU), and slope α.

As seen in Table 1, multiple bioreactors were seeded with different number of cells ranging 151 to 453 million cells per bioreactor. Three identical bioreactors (bioreactor #1, #2, and #3) had the same packed bed height (2.7 cm), were seeded with the same number of cells (151 million cells per bioreactor), and had the same total packed bed surface area (6780 cm²) and seeding density (22,222 cells/cm²). Three other identical bioreactors (#4, #5, and #6) had the same packed bed height (5.4 cm), were seeded with the same number of cells (302 million cells per bioreactor), and had the same total packed bed surface area (13,560 cm²) and seeding density (22,227 cells/cm²). A final bioreactor (#7) had an increased bed height (8.1 cm), total cells seeded (453 million cells), and packed bed surface area (20,340 cm²), but a similar seeding density (22,222 cells/cm²). During the five-day culture process, bulk media conditions (pH, DO₂, temperature, and CO₂) were maintained in automatic mode by a control system according to one or more embodiments described herein. The control system operated the media conditioning vessel to maintain media conditions, with FIGS. 4A-4C representing typical measurements of the controlled media. The bioreactor system's media perfusion flow rate was maintained automatically to maintain DO₂ at the bioreactor outlet at a specific saturation level. Again, the graph shown in FIG. 3 is typical of the perfusion flow and media outlet DO₂ found during these experiments. From graphs such as those in FIG. 3, the value of total oxygen consumption was derived, similar to that shown in FIG. 5. The slope of the linear curve fit of those graphs (like a in FIG. 5) was determined for each bioreactor run and is presented in last column of Table 1 (slope α). The value of the slope α determined as described above can be used as process analytical tool to control and describe upstream bioprocess and predict the biomass production inside the packed bed matrix.

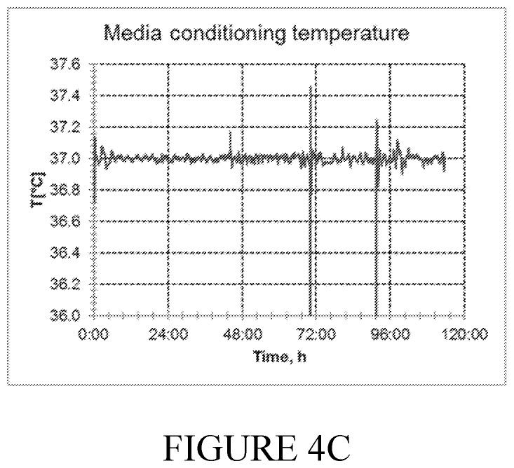

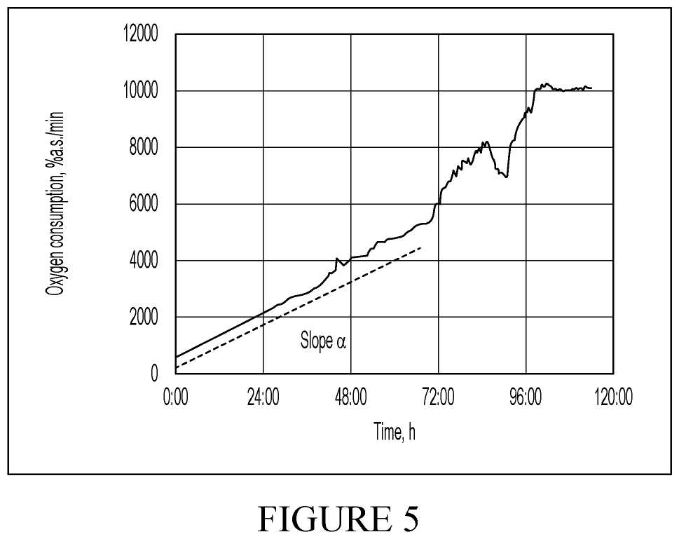

For example, FIGS. 6, 7, and 8 plot the values of a in Table 1 against the seeded cell number, packed bed surface area, and harvest density, respectively. The linearity of these graphs can be used to predict cell culture response according to various cell culture system parameters. For instance, the linearity of the graph in FIG. 6 indicates that upstream processes developed for small scale bioreactors #1 and #2 in Table 1 can be scaled 2× and 3× for bioreactors #4-7. Thus, constant monitoring and logging of the slope α value can serve for determining scalability of the upstream process. An alternative way to verify process scalability is to plot slope α relative to the surface area of bioreactor, as shown in FIG. 7. The orange data point in FIGS. 6 and 7 corresponds to failed bioreactor #3 from Table 1 (discussed below).

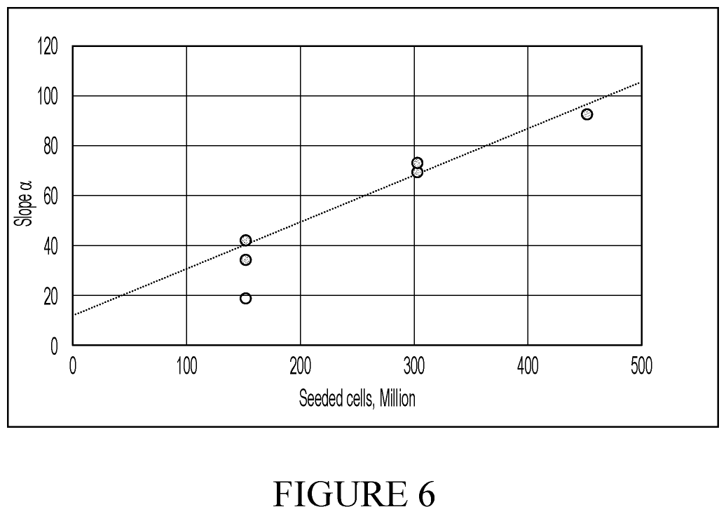

Monitoring of the slope α value during a bioreactor run serves as the characteristic signal signature that reflects health and expansion of cells culture. For example, as indicated in Table 1, bioreactors #1, 2 and 3 were seeded with the same number of cells. The characteristic signal signatures (slope α) were measured for all bioreactors. FIG. 8 indicates that real time monitoring of slope α can be used to compare performance of identical bioreactors and predict bioreactor productivity. From FIG. 8, it can be seen that bioreactor #3 run was in suboptimal conditions that resulted in lowest cells yield. Therefore, monitoring the value of slope α during bioprocess run can be used as characteristic signal signature for a given process and can detect any process deviation if the predetermined value is not within the range that was defined during process development optimization.

According to some embodiments, the media conditioning vessel is controlled by the controller to provide the proper temperature, pH, O₂, and nutrients. While in some embodiments, the bioreactor can also be controlled by the controller, in other embodiments the bioreactor is provided in a separate perfusion circuit, where a pump is used to control the flow rate of media through the perfusion circuit based on the detection of O2 at or near the outlet of the bioreactor.

The cell culture matrix can be arranged in multiple configurations within the culture chamber depending on the desired system. For example, in one or more embodiments, the system includes one or more layers of the substrate with a width extending across the width of a defined cell culture space in the culture chamber. Multiple layers of the substrate may be stacked in this way to a predetermined height. The substrate layers may be arranged such that the first and second sides of one or more layers are perpendicular to a bulk flow direction of culture media through the defined culture space within the culture chamber, or the first and second sides of one or more layers may be parallel to the bulk flow direction. In one or more embodiments, the cell culture matrix includes one or more substrate layers at a first orientation with respect to the bulk flow, and one or more other layers at a second orientation that is different from the first orientation. For example, various layers may have first and second sides that are parallel or perpendicular to the bulk flow direction, or at some angle in between.

In one or more embodiments, the cell culture system includes a plurality of discrete pieces of the cell culture substrate in a packed bed configuration, where the length and or width of the pieces of substrate are small relative to the culture chamber. As used herein, the pieces of substrate are considered to have a length and/or width that is small relative to the culture chamber when the length and/or width of the piece of substrate is about 50% or less of the length and/or width of the culture space. Thus, the cell culture system may include a plurality of pieces of substrate packed into the culture space in a desired arrangement. The arrangement of substrate pieces may be random or semi-random, or may have a predetermined order or alignment, such as the pieces being oriented in a substantially similar orientation (e.g., horizontal, vertical, or at an angle between 0° and 90° relative to the bulk flow direction).

The “defined culture space,” as used herein, refers to a space within the culture chamber occupied by the cell culture matrix and in which cell seeding and/or culturing is to occur. The defined culture space can fill approximately the entirety of the culture chamber, or may occupy a portion of the space within the culture chamber. As used herein, the “bulk flow direction” is defined as a direction of bulk mass flow of fluid or culture media through or over the cell culture matrix during the culturing of cells, and/or during the inflow or outflow of culture media to the culture chamber.

In some embodiments of this disclosure, there is only a single bulk flow direction within the defined culture space, the packed bed, and/or the bioreactor vessel, such that the liquid or media flow proceeds in predominately one direction from the bioreactor inlet through the packed bed to the bioreactor outlet. The liquid or media flow is uninterrupted by any complicated flow paths within the packed bed space and proceeds through the packed bed in predominately one direction. This avoids complicating flow paths used in some conventional bioreactors where flow spacers, separators, or channels are used to help distribute cell culture media through a cell culture substrate, often because of the inherent non-uniformity of the bioreactor or cell culture substrate. However, in embodiments of the current disclosure, such complicated flow paths are not necessary, and the media flow can be maintained in a single direction from the inlet of the bioreactor to the outlet of the bioreactor. The foregoing is not intended to preclude the use of flow distributor plates at the inlet and outlets of the bioreactor plate, which can be used to distribute fluid across a width of the bioreactor vessel and/or control pressure differentials within the reactor, but do not otherwise affect the bulk flow direction through the packed bed and/or within the cell culture space within the bioreactor vessel interior.

The packed bed cell culture matrix of one or more embodiments can include a substrate material constructed to have a uniform and ordered porous structure. The substrate may be referred to as a “structurally defined” substrate meaning that the substrate has a physical structure that is non-random, but instead is ordered according to defined parameters. In one or more embodiments, the structurally defined substrate includes a plurality of openings defining a porosity of the substrate, the plurality of openings being arrayed in a regular or uniform pattern in each substrate piece or layer. In one or more embodiments, the packed bed cell culture substrate may include a woven cell culture mesh substrate without any other form of cell culture substrate disposed in or interspersed with the cell culture matrix. That is, the woven cell culture mesh substrate of embodiments of this disclosure are effective cell culture substrates without requiring the type of irregular, non-woven substrates used in existing solution. This enables cell culture systems of simplified design and construction, while providing a high-density cell culture substrate with the other advantages discussed herein related to flow uniformity, harvestability, etc.

In one or more embodiments, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

By using a structurally defined culture matrix of sufficient rigidity, high-flow-resistance uniformity across the matrix or packed bed is achieved. According to various embodiments, the matrix can be deployed in monolayer or multilayer formats. This flexibility eliminates diffusional limitations and provides uniform delivery of nutrients and oxygen to cells attached to the matrix. In addition, the open matrix lacks any cell entrapment regions in the packed bed configuration, allowing for complete cell harvest with high viability at the end of culturing. The matrix also delivers packaging uniformity for the packed bed, and enables direct scalability from process development units to large-scale industrial bioprocessing unit. The ability to directly harvest cells from the packed bed eliminates the need of resuspending a matrix in a stirred or mechanically shaken vessel, which would add complexity and can inflict harmful shear stresses on the cells. Further, the high packing density of the cell culture matrix yields high bioprocess productivity in volumes manageable at the industrial scale.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

According to one or more embodiments the cell culture substrate can be one according to the cell culture matrices and/or substrate materials disclosed in U.S. patent application Ser. Nos. 16/781,685; 16/781,723; 16/781,764; 16/781,807; 16/781,847; 16/781,883; and Ser. No. 16/765,722, all of which are incorporated herein by reference in their entireties.

According to some embodiments, a method of cell culturing is also provided using bioreactors with the matrix for bioprocessing production of therapeutic proteins, antibodies, viral vaccines, or viral vectors.

The cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

Embodiments of this disclosure are relevant to scalable bioreactor systems, including large scale adherent cell culture fixed bed bioreactors. Fixed bed bioreactors have been increasingly used for scale-up productions of cells, viral vectors, extracellular vesicles, and therapeutical proteins. Scalability is a key aspect to advance a process from development stage to production scale. It is highly desirable to have the ability to monitor all relevant parameters with the same measurement type in each process scale to keep the product quality and product quantity high and within GMP compliance. The United States Food and Drug Administration (FDA) has recommended the adoption of process analytical technology (PAT) as a mechanism to design, analyze, and control pharmaceutical manufacturing processes through the measurement of critical process parameters which affect critical quality attributes.

The viable cell concentration (VCC), also termed biomass, is one of the most important key performance indicators during upstream technologies in mammalian cell culture. For suspension cell culture, biomass monitoring can be achieved using optical or electric approaches. However, for adherent cell culture using fixed bed bioreactors there are not validated approaches or sensors available for online biomass monitoring. In fact, for adherent cell culture, biomass is often measured by offline microscopic imaging methods after staining. Historically, substrate (e.g., glucose) consumption rates have been proposed to be useful for understanding the kinetics of cell growth. However, since substrate consumption rates are influenced by many factors besides viable cell number, there are no established protocols, methods, and/or models to obtain and use effective substrate consumption rates and metabolite accumulation rates for biomass calculation and predications.

However, embodiments of this disclosure disclose methods and protocols to obtain effective glucose consumption rate, glutamine consumption rate, lactate accumulation rate, and/or ammonia accumulation rate, and the use of at least one of these rates based on mathematic models for biomass monitoring of adherent cell culture in fixed bed bioreactors. Embodiments of the methods include (1) the use of glucose and/or glutamine rich medium, (2) measuring glucose, glutamine, lactate and/or ammonia concentrations multiple times (e.g., at least twice per day), (3) determining the rate of glucose and glutamine consumption as well as lactate and/or ammonia accumulation, and (4) use a mathematic model to calculate and predict biomass at a specific time of cell culture. Given the availability of online glucose sensors, the present methods can be used for online biomass prediction and calculation, which can be used as a guide for, e.g., transfection schedule and cell harvesting. Since medium samples can be collected using offline sampling devices such as syringes, aspects of embodiments of the methods in this disclosure can also be done offline using a multiplexing analyzer (such as, e.g., Flex II Analyzer from Nova Biomedical).

Aspects of embodiments of the present disclosure describe methods to identify any abnormality of adherent cell culture in a fixed bed bioreactor. Aspects of embodiments include comparison of glucose consumption rate or lactate accumulation rate with glutamine consumption rate or ammonia accumulation rate. According to certain aspects of embodiments, any great divergency between glucose/lactate rate and glutamine/ammonia rate is an indication of abnormality of the cell culture, such as cells undergo apoptosis when cells become overconfluent. In contrast, good agreement between glucose/lactate rate and glutamine/ammonia rate is an indication of healthy cell growth.

According to embodiments, the medium used in the cell culture preferably contains high concentration of glucose (e.g., 2 g/L, 3 g/L, 4 g/L, 5 g/L, or even 10 g/L), and/or high concentration of glutamine (e.g., 2 mM, 3 mM, 4 mM, 5 mM, or even 10 mM). In aspects of embodiments, the cell culture medium is replenished or exchanged daily, at least when viable cell concentration become high at later time of cell culture, so that the concentration of glucose and/glutamine concentration never falls below a threshold. This threshold may depend on the specifics of the cell culture, but can be, for example, 0.25 g/L for glucose and/or 0.25 mM for glutamine. It is believed that this has an impact on effective substrate consumption rate and metabolite accumulation rates measurements, because, for a given cell density at a given culture time, the glucose consumption rate will vary depending on the initial glucose concentration.

As an aspect of embodiments, the cell seeding density is pre-determined and optimized based on cell culture setup. For example, the cell seeding density may be 20000 cells/cm² surface area, in a certain cell culture setup. However, this number is provided as an example, only, as it should be understood that cell seeding needs to be optimized such that the majority of cells (e.g., >90%) become attached to fixed bed substrate surfaces.

The cell medium substrates and metabolites can be measured using an online sensor, or offline sensors or analyzer after samples are collected. The cell medium substrates and metabolites can be measured daily, according to embodiments, including, for example, at least twice per day. The two measurements can be separated by at least a specific time interval, which may be, for example, 30 min, 1 hr, 2 hrs, 3 hrs, 4 hrs, 5 hrs, 6 hrs, 7 hrs, 8 hrs, or even 20 hrs. The first measurement can be done at least one hour after medium exchange, if any, is completed.

Depending on the bioreactor setup, appropriate mathematic models are used to calculate glucose/glutamine consumption rates, and lactate/ammonia accumulation rates, and predict and calculate biomass at a specific culture duration.

### Examples

In experiments conducted based on embodiments of this disclosure, a fixed-bd bioreactor system as described herein was used in a perfusion loop with a media conditioning vessel (MCV) and pump. Two 1 mL syringes were attached to the perfusion loop tubing just upstream of the inlet 112 and downstream of the outlet 114 (see FIG. 1) of the bioreactor vessel. The syringes were used to collect medium samples every 30 minutes each day for 8 hours per day. Early in the morning of each day, half of the media in the MCV was drained and replaced back with the same volume plus 40 mL of extra media to accommodate sampling from the previous day with fresh, warmed media. All samples collected were analyzed offline using a Flex II Analyzer from Nova Biomedical. HEK293T cells were used. Cell seeding density was 22,000 cells/cm². Cell attachment was found to be completed within 3.5 hrs, with >95% of cells become attached.

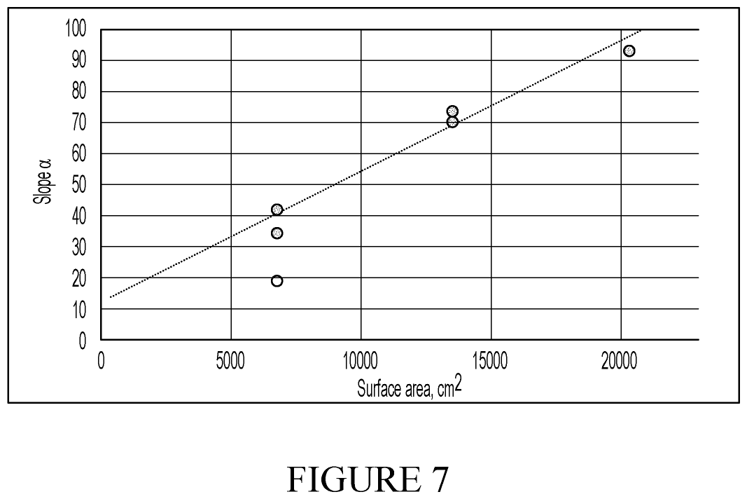

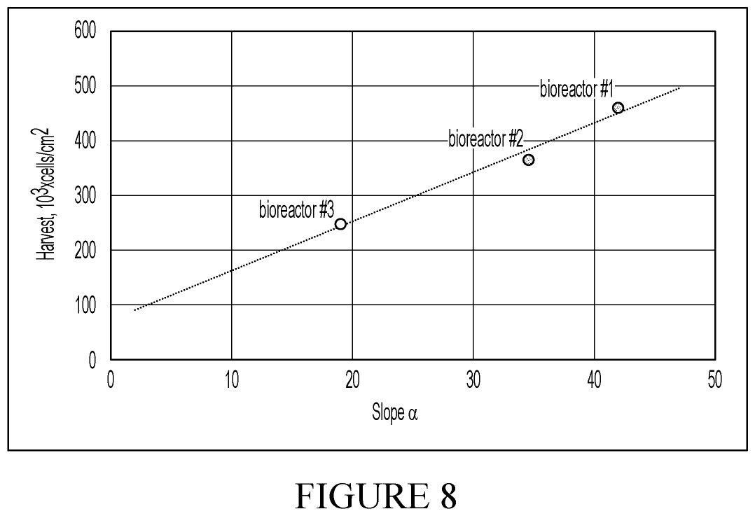

As shown in FIG. 9, the glucose concentration decreased over time each day. Similar results were obtained for glutamine. In contrast, the lactate concentration increased over time at each day, as shown in FIG. 10 (similar results were obtained for ammonia). However, the difference between the samples collected from upstream or downstream of the bioreactor at a specific culture time is very small, even in the last day of the cell culture, suggesting that the amount of glucose or glutamine consumption is relatively small when the medium pass through the bioreactor just once, which therefore may not be used to obtain effective substrate consumption or metabolite accumulation rates. Therefore, according to embodiments of this disclosure, only the concentrations of substrates or metabolites from the samples collected upstream of the inlet of the bioreactor may be used for biomass analysis.

The cell growth rate, k, is a function of many factors including medium composition and concentrations, temperature and other environmental factors such as pH, dissolved oxygen and CO₂. Assuming these environmental factors can be controlled such that cell growth rate maintains constant, one can integrate over the entire surface area A, yielding total cell count N(t):

$$N ( t ) = {N}_{0} {e}^{kt} \qquad Equation ( 2 )$$

wherein N₀ is the initial cell number.

According to a mass balance perfusion reactor model, the cell medium substrate concentration gradient Cᵢₙ-Cₒᵤₜ, when the cell medium passes through the bioreactor, is a function of the medium substrate consumption rate r per cell, total cell number N and flow rate Q:

$${C}_{in} - {C}_{out} = \frac{rN}{Q} \qquad Equation ( 3 )$$

In Equation (3), Cᵢₙ is the concentration of a cell medium substrate entering the bioreactor, while Cₒᵤₜ is the concentration of the cell medium substrate exiting the bioreactor and Q is the flow rate. As mentioned above, in this experimental setup the flow rate was about 50 mL/min, such that the cell medium substrate concentration displayed little or very small changes when the medium just passes through the bioreactor. Therefore, it is difficult or impossible to directly use these concentration gradients to calculate biomass. In order to use this parameter to calculate biomass, one would need to reduce the flow rate dramatically, or stop the flow temporarily (for example, 1 min, 2 min, 10 min, etc.) so that the sensor can detect the meaningful difference between Cᵢₙ and Cₒᵤₜ for a given analyte. For instance, for HEK293T cell culture, when the cell number is 1×10⁹ cells within 1 m² bioreactor where about 100 mL medium is required to fill the entire bioreactor, one would need stop the flow for 10 minutes to effectively detect 0.1 g/L of glucose consumed, assuming the glucose consumption rate is about 0.001 mg/min/Million of cells. Such low flow rate or flow stopping for a period of time not only is difficult to be achieved practically, but also would cause temporary hypoxia culture condition.

According to a continuously stirred tank reactor model for MCV which is coupled with the bioreactor, the total amount of cell medium substrate consumed during a given period of time is a function of the cell consumption rate r and total cell number N:

$$- \frac{d ( VC )}{dt} = Q ( {C}_{in} - {C}_{out} ) = r N \qquad Equation ( 4 )$$

wherein V is the total volume of medium in the MCV. After re-arrangement:

$$- \frac{dC}{dt} = \frac{rN}{V} - dC = \frac{r N}{V} dt = \frac{r {N}_{0}}{V} {e}^{kt} dt - {\int }_{{C}_{0}}^{C} d C = \frac{r {N}_{0}}{V} {\int }_{0}^{t} {e}^{kt} dt {C}_{0} - C ( t ) = \frac{r {N}_{0}}{V} \frac{1}{k} ( {e}^{kt} - 1 ) \qquad Equation ( 5 )$$

Since kt is small enough within a maximum of the 8 hours sampling window each day (<0.5), thus:

$${C}_{0} - C ( t ) = \frac{r {N}_{0}}{V} \frac{1}{k} ( {e}^{kt} - 1 ) \approx \frac{r {N}_{0}}{V} \frac{1}{k} ( 1 + kt - 1 ) = \frac{r {N}_{0}}{V} t V ( {C}_{0} - C ( t ) ) = r {N}_{0} t \qquad Equation ( 6 )$$

Therefore, a plot of Equation (6) (V(C₀−C) vs. t) to fit a line (time is shifted to the first meaningful sampling each day), gives a slope of rN₀, where N₀ is the total cell count at the first meaningful sampling of each day. Furthermore, the plot of rN₀ vs. t and fit to an exponential curve provides:

$$r {N}_{0} ( t ) = r {N}_{seed} {e}^{kt} \qquad Equation ( 7 )$$

The slope is the cell growth rate k, and the intercept is r*N_{seed}.

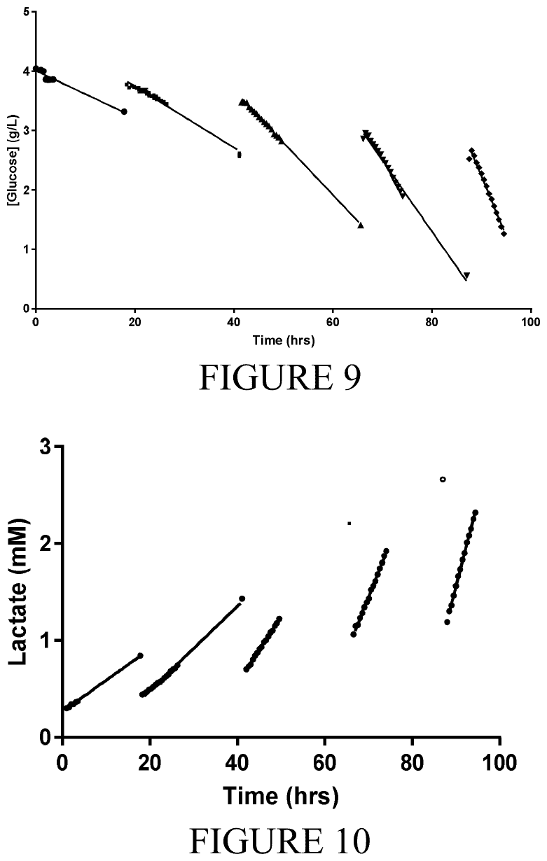

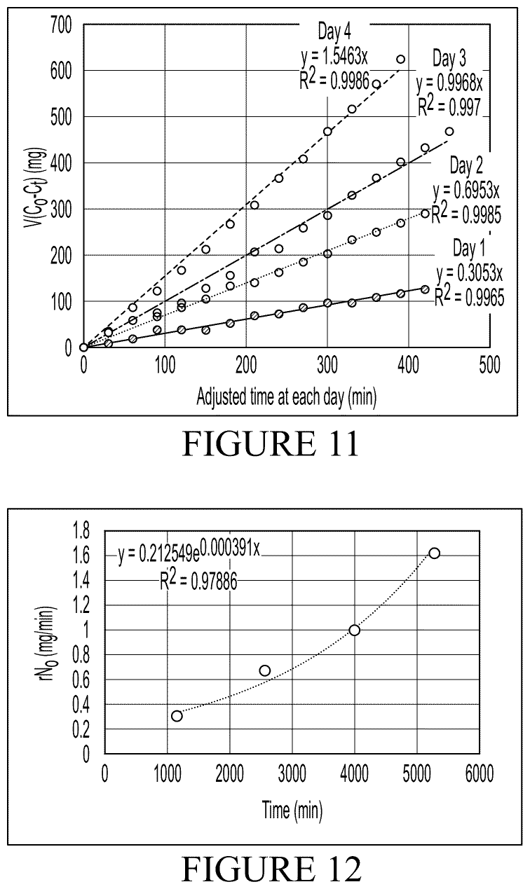

FIG. 11 shows a graph of the total amount of glucose consumed, V(C₀−Cₜ), as a function of time each day, according to example embodiments. FIG. 12 shows a graph of the total glucose consumption rate, rN₀, at each day as an exponential function over culture duration, according to example embodiments. Using glucose consumption as an example, as shown in FIG. 11, the total amount of glucose consumed increased linearly over time within each day. However, the slope, rN₀, increased daily, and fits well with an exponential function (FIG. 12), yielding a cell growth rate k of 0.000391 min⁻¹, and an intercept r*N_{seed} of 0.212549. These results suggest that the cell doubling time is about 29.5 hrs, and the glucose consumption rate is 0.0009531 mg/min/millions of cells. Therefore, we can predict the cell number at harvest time tₕ=5669 min is:

$${N}_{harvest} = {N}_{seed} {e}^{{kt}_{h}} = 2046 M \qquad Equation ( 8 )$$

The above number of 2046M is higher than the actual total cell number harvested, which was found to be 1790M.

Similarly, glutamine, lactate and ammonia profiles were also obtained and used to analyze cell growth and predict cell numbers at time of harvesting (Table 2). These results suggest that there is good agreement in cell growth rate and cell numbers at time of harvesting among all four analytes. Interestingly, both glucose and lactate gave rise to almost identical cell growth parameters, while glutamine shared similarity with ammonia. It is believed that this is because lactate is a cellular metabolite of glucose, while ammonia is a cellular metabolite of glutamine. Also notable is that glucose/lactate profiling gave rise to higher cell numbers predicted than glutamine/ammonia profiling did.

TABLE 2
Comparison of cell growth parameters
obtained using four different analytes.

| Cell growth | | | | |
|---|---|---|---|---|
| parameter | Glucose | Glutamine | Lactate | Ammonia |
| Cell growth | 0.0234 | 0.0213 | 0.022 | 0.0198 |
| rate k (hr⁻¹) | | | | |
| Cell doubling | 29.5 | 32.5 | 30.2 | 35 |
| time (hr) | | | | |
| Consumption or | 0.000953 | 0.000907 | 0.0008085 | 0.000486 |
| accumulation | mg/min/M | μmole/min/M | mg/min/M | μmole/min/M |
| rate per million | | | | |
| of cells M | | | | |
| Cell numbers | 2.046E+09 | 1.68E+09 | 1.955E+09 | 1.45E+09 |
| at time of | | | | |
| harvesting | | | | |

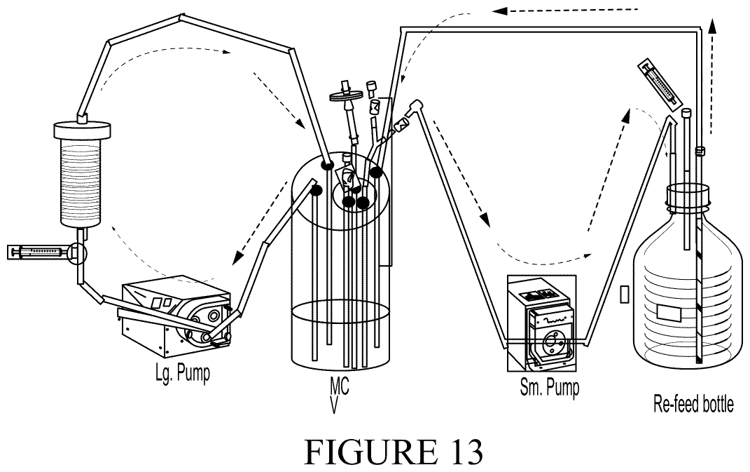

Additional experiments were carried out using an alternative bioreactor system setup that included a re-feed bottle, as shown in FIG. 13. One syringe was connected to the tubing right below the inlet of the bioreactor, where the substrate concentration is termed Cₜ. The second syringe is connected to the contents of the re-feed bottle, where the substrate concentration is termed C_{2,t}. Two independent bioreactors with a substrate surface area of 5 m² were assessed. Both used 22,000 cells/cm² as the seeding density, and cultured for 4 days for HEK293T cells. The MCV contained 1800 mL media, while the re-feed loop contained 3 L media. For cell seeding, 300 mL of cell solution was used. Results showed that cells achieved ~95% attachment within 3.5 hrs. Each day, 2 mL samples were collected from the under-reactor port and the re-feed bottle at least twice. At day 2, there was a medium exchange in the afternoon; at day 3, there was two medium exchange, one in early morning, and another late afternoon. The cells were harvested at early morning of day 4 using an auto harvest protocol. All samples collected were analyzed using the Nova Biomedical Flex II Analyzer.

Since this bioreactor setup involves a third re-feed bottle, a modified model is used to analyze the profiles. Let V₂ and C₂ be the media volume and glucose concentration in the re-feed bottle, and Q₂ is the flow rate between MCV and re-feed bottle, then for the MCV:

$$\frac{d ( VC )}{dt} = - r N + {Q}_{2} ( {C}_{2} - C )$$

For the re-feed bottle:

$$\frac{d ( {V}_{2} {C}_{2} )}{dt} = {Q}_{2} ( C - {C}_{2} )$$

Substituting it into the MCV equation:

$$\frac{d ( VC + {V}_{2} {C}_{2} )}{dt} = - rN$$

Then the solution is:

$$V ( {C}_{0} - C ( t ) ) + {V}_{2} ( {C}_{2 , 0} - {C}_{2} ( t ) ) = {rN}_{0} t$$

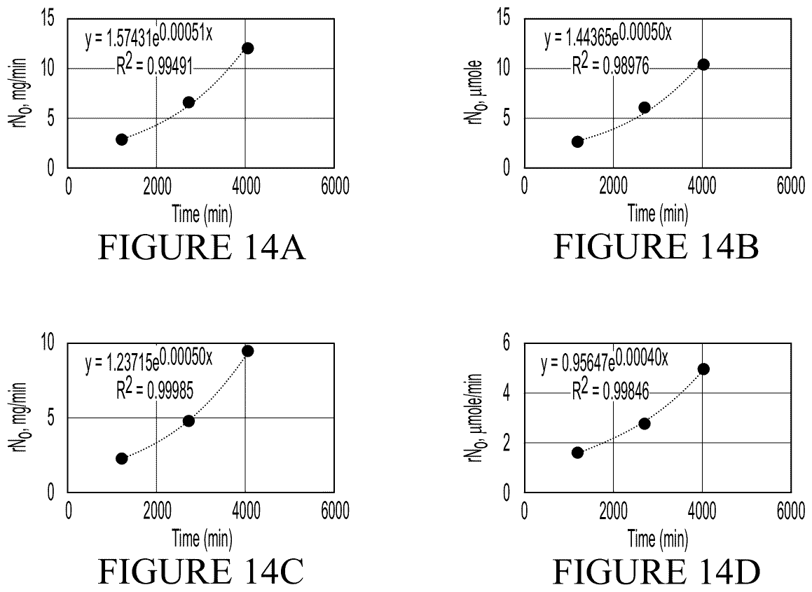

Glucose, glutamine, lactate and ammonia profiles were also obtained and used to analyze cell growth and predict cell numbers at time of harvesting. As exampled for the first bioreactor (FIG. 14A-14D), for all four analyte profiles their corresponding consumption rate or accumulation rate fits well with an exponential function, consistent with the exponential growth pattern of typical adherent cell culture. Further analysis also suggested that there is good agreement in cell growth rate and cell numbers at time of harvesting among all four analytes (Table 3). Again, both glucose and lactate gave rise to almost identical cell growth parameters, while glutamine shared similarity with ammonia. The different cell growth parameters obtained may be related to slightly different culture conditions or cell culture variabilities from bioreactor to bioreactor (e.g., compare Table 3 results for the first bioreactor with Table 4 results for the second bioreactor). In general, the cell parameters predicted using ammonia accumulation rate are lower than all other three analytes, possibly due to the unique properties of ammonia. It is known that some of ammonia produced escapes from the cell culture medium and enters into head space of the MCV.

TABLE 3
Comparison of cell growth parameters obtained using four different
analytes for the first 5m² bioreactor.

| Cell growth | | | | |
|---|---|---|---|---|
| parameter | Glucose | Glutamine | Lactate | Ammonia |
| Cell growth | 0.0312 | 0.0306 | 0.0306 | 0.0246 |
| rate k (hr⁻¹) | | | | |
| Cell doubling | 22.2 | 22.6 | 22.6 | 28.2 |
| time (hr) | | | | |
| Consumption or | 0.0014 | 0.0013 | 0.0011 | 0.0008 |
| accumulation | mg/min/M | μmole/min/M | mg/min/M | μmole/min/M |
| rate per million | | | | |
| of cells M | | | | |
| Cell numbers | 1.77E+10 | 1.68E+10 | 1.68E+10 | 9.3E+09 |
| at time of | | | | |
| harvesting | | | | |

| | |
|---|---|
| Actual cell | 1.9E+10 |
| numbers at | |
| time of harvest | |

TABLE 4
Comparison of cell growth parameters obtained using four different
analytes for the second 5m² bioreactor.

| Cell growth | | | | |
|---|---|---|---|---|
| parameter | Glucose | Glutamine | Lactate | Ammonia |
| Cell growth | 0.036 | 0.0324 | 0.034 | 0.026 |
| rate k (hr⁻¹) | | | | |
| Cell doubling | 19.2 | 21.4 | 20.2 | 26.9 |
| time (hr) | | | | |
| Consumption or | 0.000739 | 0.000828 | 0.000705 | 0.000709 |
| accumulation | mg/min/M | μmole/min/M | mg/min/M | μmole/min/M |
| rate per million | | | | |
| of cells M | | | | |
| Cell numbers | 2.70E+10 | 1.96E+10 | 2.30E+10 | 1.09E+10 |
| at time of | | | | |
| harvesting | | | | |

| | |
|---|---|
| Actual cell | 1.95E+10 |
| numbers at | |
| time of harvest | |

As an alternative method, according to some embodiment, a metabolite kinetics model can be used. The metabolite kinetics model above requires that for each data set used to fit V(C₀−C(t))=rN₀t or V(C₀−C(t))+V₂(C_{2,0}−C₂(t))=rN₀t, there is no media addition or exchange taking place during the time span of the data set. This kinetics model has an advantage in that the operator does not need know the history of operation throughout the culture, as long as there is no media addition or exchange taking place during the time span of each data set collected.

In reality, however, it is not uncommon that there are only a limited number of samplings spanned across several days, with media additions or exchanges in between, sometime with high frequency. For instance, in the late phase of cell culture the cell confluency may become high so it is common to add a fixed amount of medium periodically (e.g., every one hour) to maintain relatively constant medium compositions, thereby improving productivity of culture. In such scenario, an alternative approach is provided as follows:

$${Δ ( VC )}_{adjusted} = \frac{r {N}_{0}}{k} ( {e}^{kt} - 1 )$$

Where Δ(VC)_{adjusted} stands for the change of metabolite amount in the MCV from t=0, adjusted according to any media addition or exchange event. This model is referred to the accumulative model.

Table 5 provides an example. Suppose there are 7 samplings starting with t=0, and there is one media addition with volume of Va after t2, and one media exchange with volume of Ve after t4. Δ(VC) is calculated as before, but needs to be adjusted for all samplings after any media addition/exchange event. For example, for the media addition after t2, Δ(VC) is added by Va*Ca for all samplings since t3 (Ca is the fresh media concentration). Similarly, for the media exchange after t4, Δ(VC) needs to be adjusted by Ve*(Ca−Ce) since t5, where Ce is the concentration of media removed. To avoid the need of sampling the media removed, it is suggested to perform the media exchange right after a regular sampling—in this case, right after t4 so that Ce=C4.

TABLE 5
An example of data pre-processing for the alternative model.

| t | V | C | Δ(VC) | Δ(VC)adjusted |
|---|---|---|---|---|
| 0 | V0 | CO | 0 | 0 |
| t1 | V1 = V0 | C1 | V0*C0-V1*C1 | (V0*CO-V1*C1) |
| t2 | V2 = V1 | C2 | V0*C0-V2*C2 | (V0*C0-V2*C2) |
| Media addition | Va | | | |
| t3 | V3 = V2 + Va | C3 | V0*C0-V3*C3 | (V0*C0-V3*C3) + |
| | | | | Va*Ca |
| t4 | V4 = V3 | C4 | V0*C0-V4*C4 | (V0*CO-V4*C4) + |
| | | | | Va*Ca |
| Media exchange | Ve | | | |
| t5 | V5 = V4 | C5 | V0*C0-V5*C5 | (V0*C0-V5*C5) + |
| | | | | Va*Cf + |
| | | | | Ve*(Ca-Ce) |
| t6 | V6 = V5 | C6 | V0*C0-V6*C6 | (V0*C0-V6*C6) + |
| | | | | Va*Cf + |
| | | | | Ve*(Ca-Ce) |

In the re-feed bottle experiment above, there were media exchanges from the system. Using the method suggested above, we can prepare the data for the model:

$${Δ ( VC )}_{adjusted} = \frac{r {N}_{0}}{k} ( {e}^{kt} - 1 )$$

Note that in this case Δ(VC)_{adjusted} has the physical meaning of total glucose consumed.

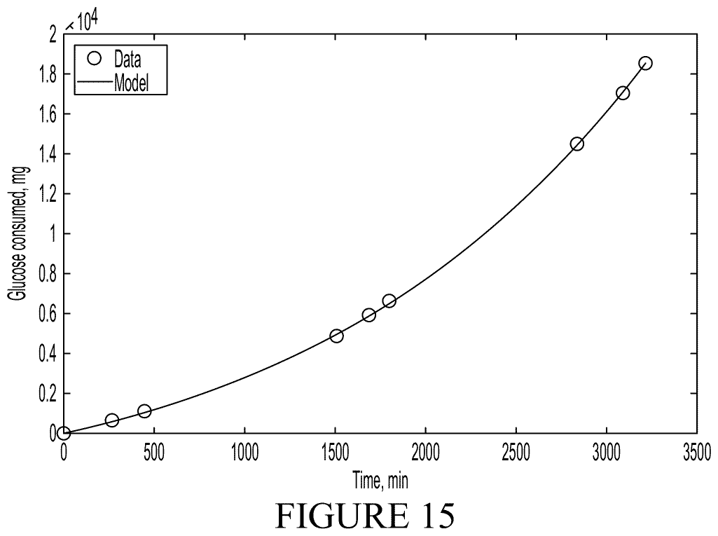

FIG. 15 shows that the total glucose consumption (correlated to the total cell number) has a good fit to the exponential function in the model. The predicted cell growth constant k is 5.44e-04 min⁻¹ (doubling time 21 hr). The predicted cell yield is 19893 M, in close agreement to the harvest data which was 19,175M.

As demonstrated, this accumulative model has an advantage that it is suitable for a wide range of cell culture protocols as well as metabolite data collection protocols, including fewer samplings spanned with longer intervals, and frequent medium operation. However, this model needs to track the entire history of the operation of the cell culture, in particular any medium addition, removal, or exchange.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.
Aspect 1 pertains to a method of monitoring biomass during a cell culture of cells in a bioreactor, the method comprising: culturing the cells in the bioreactor using a cell culture medium perfused through the bioreactor; measuring at least one of a cell nutrient and a cell byproduct in the cell culture medium; determining at least one of a consumption rate of the cell nutrient and an accumulation rate of the cell byproduct; predicting a cell number within the bioreactor at a specified culture time based on at least one of the consumption rate and the accumulation rate.
Aspect 2 pertains to the method of Aspect 1, wherein the bioreactor is a fixed bed bioreactor comprising a substrate configured for culturing cells attached to a surface of the substrate.
Aspect 3 pertains to the method of Aspect 1 or Aspect 2, wherein the at least one cell nutrient is glucose or glutamine.
Aspect 4 pertains to the method of any one of Aspects 1-3, wherein the at least one cell byproduct is lactate or ammonia.
Aspect 5 pertains to the method of any one of Aspects 1-4, wherein the cell culture medium is a glucose- or a glutamine-rich cell culture medium.
Aspect 6 pertains to the method of any one of Aspects 1-5, wherein the measuring the at least one of the cell nutrient and the cell byproduct in the cell culture medium comprises taking multiple measurements of the cell nutrient or the cell byproduct, the multiple measurements being separated by a measurement interval that is less than a doubling time of the cells in the cell culture.
Aspect 7 pertains to the method of Aspect 6, wherein the measurement interval is greater than or equal to a minimum interval time, the minimum interval time being a time at which the change in the level of the cell nutrient or the cell byproduct is larger than a measurement tolerance of measuring the cell nutrient or cell byproduct.
Aspect 8 pertains to the method of Aspect 7, wherein the minimum interval time is greater than or equal to about 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, or 20 hours.
Aspect 9 pertains to the method of any one of Aspects 1-8, wherein the measuring of the cell nutrient in the cell culture medium comprises measuring the cell nutrient multiple times per day of the cell culture.
Aspect 10 pertains to the method of any one of Aspects 1-9, wherein the measuring of the cell byproduct in the cell culture medium comprises measuring the cell byproduct multiple times per day of the cell culture.
Aspect 11 pertains to the method of any one of Aspects 1-10, wherein the predicting the cell number comprises using a mathematical model to calculate biomass at the specified culture time.
Aspect 12 pertains to the method of any one of Aspects 1-11, wherein the measuring comprises using an inline sensor in a perfusion line of the cell culture medium.
Aspect 13 pertains to the method of any one of Aspects 1-11, wherein the measuring comprises using an offline measurement of samples of the cell culture medium.
Aspect 14 pertains to the method of any one of Aspects 1-13, further comprising, after the determining of the consumption rate and the accumulation rate, comparing at least one of the consumption rate of a first cell nutrient and the accumulation rate of a first cell byproduct to at least one of the consumption rate of a second cell nutrient and the accumulation rate of a second cell byproduct.
Aspect 15 pertains to the method of Aspect 14, wherein the comparing comprises comparing the consumption rate of glucose to the consumption rate of glutamine.
Aspect 16 pertains to the method of Aspect 14 or Aspect 15, wherein the comparing comprises comparing the consumption rate of glucose to the accumulation rate of ammonia.
Aspect 17 pertains to the method of any one of Aspects 14-16, wherein the comparing comprises comparing the accumulation rate of lactate to the consumption rate of glutamine.
Aspect 18 pertains to the method of any one of Aspects 14-17, wherein the comparing comprises comparing the accumulation rate of lactate to the accumulation rate of ammonia.
Aspect 19 pertains to the method of any one of Aspects 14-18, further comprising determining an abnormality in the cell culture based on the comparing.
Aspect 20 pertains to the method of any one of Aspects 1-19, further comprising seeding cells in the bioreactor at a seeding density.
Aspect 21 pertains to the method of any one of Aspects 1-20, further comprising supplying fresh cell culture medium to the bioreactor.
Aspect 22 pertains to the method of Aspect 21, wherein the measuring comprising a first measurement, the first measurement occurring at least one hour after supplying the fresh cell culture medium.
Aspect 23 pertains to the method of any one of Aspects 1-22, wherein the predicting of the cell number, Nₜ, comprises using the following equation:

$${N}_{t} = {N}_{seed} {e}^{kt}$$

where N_{seed} is the number of cells seeded into the bioreactor, k is a cell growth rate, and t is the time for which the cell number is being predicted.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A method of monitoring biomass during a cell culture of cells in a bioreactor, the method comprising:
culturing the cells in the bioreactor using a cell culture medium perfused through the bioreactor;
measuring at least one of a cell nutrient and a cell byproduct in the cell culture medium;
determining at least one of a consumption rate of the cell nutrient and an accumulation rate of the cell byproduct;
predicting a cell number within the bioreactor at a specified culture time based on at least one of the consumption rate and the accumulation rate,
wherein the measuring the at least one of the cell nutrient and the cell byproduct in the cell culture medium comprises taking multiple measurements of the cell nutrient or the cell byproduct, the multiple measurements being separated by a measurement interval that is less than a doubling time of the cells in the cell culture.

2. The method of claim 1, wherein the bioreactor is a fixed bed bioreactor comprising a substrate configured for culturing cells attached to a surface of the substrate.

3. The method of claim 1, wherein the at least one cell nutrient is glucose or glutamine.

4. The method of claim 1, wherein the at least one cell byproduct is lactate or ammonia.

5. (canceled)

6. (canceled)

7. The method of claim 1, wherein the measurement interval is greater than or equal to a minimum interval time, the minimum interval time being a time at which the change in the level of the cell nutrient or the cell byproduct is larger than a measurement tolerance of measuring the cell nutrient or cell byproduct.

8. The method of claim 7, wherein the minimum interval time is greater than or equal to about 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, or 20 hours.

9. (canceled)

10. (canceled)

11. The method of claim 1, wherein the predicting the cell number comprises using a mathematical model to calculate biomass at the specified culture time.

12. The method of claim 1, wherein the measuring comprises using an inline sensor in a perfusion line of the cell culture medium.

13. The method of claim 1, wherein the measuring comprises using an offline measurement of samples of the cell culture medium.

14. The method of claim 1, further comprising, after the determining of the consumption rate and the accumulation rate, comparing at least one of the consumption rate of a first cell nutrient and the accumulation rate of a first cell byproduct to at least one of the consumption rate of a second cell nutrient and the accumulation rate of a second cell byproduct.

15. The method of claim 14, wherein the comparing comprises comparing the consumption rate of glucose to the consumption rate of glutamine.

16. The method of claim 14, wherein the comparing comprises comparing the consumption rate of glucose to the accumulation rate of ammonia.

17. The method of claim 14, wherein the comparing comprises comparing the accumulation rate of lactate to the consumption rate of glutamine.

18. The method of claim 14, wherein the comparing comprises comparing the accumulation rate of lactate to the accumulation rate of ammonia.

19. The method of claim 14, further comprising determining an abnormality in the cell culture based on the comparing.

20. (canceled)

21. The method of claim 1, further comprising supplying fresh cell culture medium to the bioreactor.

22. The method of claim 21, wherein the measuring comprising a first measurement, the first measurement occurring at least one hour after supplying the fresh cell culture medium.

23. The method of claim 1, wherein the predicting of the cell number, Nₜ, comprises using the following equation:
$${N}_{t} = {N}_{seed} {e}^{kt}$$
where N_{seed} is the number of cells seeded into the bioreactor, k is a cell growth rate, and t is the time for which the cell number is being predicted.

24. The method of claim 1, wherein the measuring of the at least one of the cell nutrient and the cell byproduct in the cell culture medium comprises measuring at least one of glucose, lactate, glutamine, and ammonia concentrations at least twice per day during the cell culture for at least three days, wherein the measurements are separated by a predetermined time interval.

25. The method of claim 24, wherein the measuring comprising measuring at least two of glucose, lactate, glutamine, and ammonia concentrations.
